(19) United States
(12) Patent Application Publication
(10) Pub. No.: US 2008/0200704 A1
(43) Pub. Date: Aug. 21, 2008

(54) PREPARATION OF AMINO ACID-FATTY ACID AMIDES

(75) Inventors: Shan Chaudhuri, Mississauga (CA); Joseph MacDougall, Mississauga (CA); Jason Peters, Mississauga (CA); James Ramsbottom, Mississauga (CA)

Correspondence Address: TORYS LLP, 79 WELLINGTON ST. WEST, SUITE 3000, TORONTO, ON, M5K 1N2, CA

(73) Assignee: MULTI FORMULATIONS LTD., Mississauga (CA)

(21) Appl. No.: 11/855,360

(22) Filed: Sep. 14, 2007

Related U.S. Application Data
(63) Continuation-in-part of application No. 11/676,630, filed on Feb. 20, 2007, now Pat. No. 7,319,157.

(51) Int. Cl. C07C 233/01

(52) U.S. Cl. 554/35

(57) ABSTRACT

The present invention describes compounds produced from an amino acid molecule and a fatty acid molecule. The compounds being in the form of amino-fatty acid compounds being bound by an amide linkage, or mixtures thereof made by reacting amino acids or derivatives thereof with an appropriate fatty acid previously reacted with a thionyl halide. The administration of such molecules provides supplemental amino acids with enhanced bioavailability and the additional benefits conferred by the specific fatty acid.

### CROSS-REFERENCE TO RELATED APPLICATION

The present application is a Continuation-in-Part of U.S. patent application Ser. No. 11/676,630 entitled “Creatine-Fatty Acids,” filed Feb. 20, 2007, and claims benefit of priority thereto; the disclosure of which is hereby fully incorporated by reference.

### FIELD OF THE INVENTION

The present invention relates to structures and synthesis of amino acid-fatty acid compounds bound via an amide linkage. Specifically, the present invention relates to a compound comprising an amino acid bound to a fatty acid, wherein the fatty acid is preferably a saturated fatty acid and bound to the amino acid via an amide linkage.

### BACKGROUND OF THE INVENTION

Participation in sports at any level either professional or amateur requires an athlete to strive to bring their bodies to a physical state which is considered optimum for the sport of interest. One of the factors that correlate positively with successful participation in a sport is a high degree of development of the aerobic capacity and/or strength of skeletal muscle. Consequently, it is important that nutrients and other requirements of muscles be readily available and that these nutrients be transported to areas where they are needed free from obstructions.

Strength and aerobic capacity are both functions of training and of muscle mass. As such, an athlete who can train harder and longer is often considered to be the most effective at participation in the sport of interest. Strenuous exercise is an effective stimulus for protein synthesis. However, muscle requires a large array of nutrients, including amino acids, in order to facilitate this increased level of protein synthesis.

Following periods of strenuous exercise, muscle tissue enters a stage of rapid nitrogen absorption in the form of amino acids and small peptides. This state of increased nitrogen absorption is a result of the body repairing exercise-induced muscle fiber damage as well as the growth and formation of new muscle fibers. It is important that muscles have sufficient levels of nitrogen, in the form of amino acids and small peptides, during this period of repair and growth. When an athlete is participating in a strenuous exercise regime and fails to ingest enough nitrogen, e.g. amino acids, the body often enters a state of negative nitrogen balance. A negative nitrogen balance is a state in which the body requires more nitrogen, to facilitate repair and growth of muscle, than is being ingested. This state causes the body to catabolize muscle in order to obtain the nitrogen required, and thus results in a decrease in muscle mass and/or attenuation of exercise-induced muscle growth. Therefore, it is important that athletes ingest adequate amounts of amino acids in order to minimize the catabolism of muscle in order to obtain the results desired from training.

Although supplementation with amino acids are quite common, the uptake of amino acids by cells is limited or slow since amino acid residues are not soluble or only slightly soluble in nonpolar organic solution, such as the lipid bilayer of cells. As a result amino acids must be transported into cells via transport mechanisms which are specific to the charges that the amino acid bears. It is therefore desirable to provide, for use in individuals, e.g. animals and humans, forms and derivatives of amino acids with improved characteristics that result in increased stability and increased uptake by cells. Furthermore, it would be advantageous to do so in a manner that provides additional functionality as compared to amino acids alone.

Fatty acids are carboxylic acids, often containing a long, unbranched chain of carbon atoms and are either saturated or unsaturated. Saturated fatty acids do not contain double bonds or other functional groups, but contain the maximum number of hydrogen atoms, with the exception of the carboxylic acid group. In contrast, unsaturated fatty acids contain one or more double bonds between adjacent carbon atoms, of the chains, in cis or trans configuration.

The human body can produce all but two of the fatty acids it requires, thus, essential fatty acids are fatty acids that must be obtained from food sources due to an inability of the body to synthesize them, yet are required for normal biological function. The fatty acids which are essential to humans are linoleic acid and α-linolenic acid.

Examples of saturated fatty acids include, but are not limited to myristic or tetradecanoic acid, palmitic or hexadecanoic acid, stearic or octadecanoic acid, arachidic or eicosanoic acid, behenic or docosanoic acid, butyric or butanoic acid, caproic or hexanoic acid, caprylic or octanoic acid, capric or decanoic acid, and lauric or dodecanoic acid, wherein the aforementioned comprise from at least 4 carbons to 22 carbons in the chain.

Examples of unsaturated fatty acids include, but are not limited to oleic acid, linoleic acid, linolenic acid, arachidonic acid, palmitoleic acid, eicosapentaenoic acid, docosahexaenoic acid and erucic acid, wherein the aforementioned comprise from at least 4 carbons to 22 carbons in the chain.

Fatty acids are capable of undergoing chemical reactions common to carboxylic acids. Of particular relevance to the present invention are the formation of amides and the formation of esters.

### SUMMARY OF THE INVENTION

In the present invention, compounds are disclosed, where the compounds comprise an amino acid bound to a fatty acid, via an amide linkage, and having a structure of Formula 1:

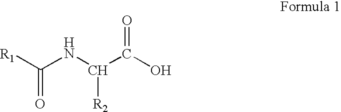

wherein:

R₁ is an alkyl group, preferably saturated, and containing from about 3 to a maximum of 21 carbons.

R₂ is hydrogen, methyl, isopropyl, isobutyl, sec butyl,

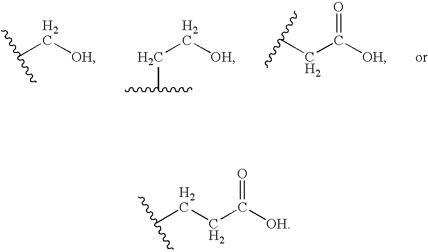

Another aspect of the invention comprises the use of a saturated fatty acid in the production of compounds disclosed herein.

A further aspect of the present invention comprises the use of an unsaturated fatty in the production of compounds disclosed herein.

### DETAILED DESCRIPTION OF THE INVENTION

In the following description, for the purposes of explanation, numerous specific details are set forth in order to provide a thorough understanding of the present invention. It will be apparent, however, to one skilled in the art that the present invention may be practiced without these specific details.

The present invention relates to structures and synthesis of amino acid-fatty acid compounds bound via an amide linkage. In addition, specific benefits are conferred by the particular fatty acid used to form the compounds in addition to, and separate from, the amino acid substituent.

As used herein, the term ‘fatty acid’ includes both saturated, i.e. an alkane chain as known in the art, having no double bonds between carbons of the chain and having the maximum number of hydrogen atoms, and unsaturated, i.e. an alkene or alkyne chain, having at least one double or alternatively triple bond between carbons of the chain, respectively, and further terminating the chain in a carboxylic acid as is commonly known in the art, wherein the hydrocarbon chain is greater than four carbon atoms. Furthermore, essential fatty acids are herein understood to be included by the term ‘fatty acid’.

As used herein, “amino acid” refers a compound consisting of a carbon atom to which are attached a primary amino group, a carboxylic acid group, a side chain, and a hydrogen atom. For example, the term “amino acid” includes, but is not limited to, Glycine, Alanine, Valine, Leucine, Isoleucine, Serine, Threonine, Aspartic acid and Glutamic acid. Additionally, as used herein, “amino acid” also includes derivatives of amino acids such as esters, and amides, and salts, as well as other derivatives, including derivatives having pharmacoproperties upon metabolism to an active form.

According to the present invention, the compounds disclosed herein comprise an amino acid bound to a fatty acid, wherein the fatty acid is preferably a saturated fatty acid. Furthermore, the amino acid and fatty acid are bound via an amide linkage and having a structure according to that of Formula 1. The aforementioned compound being prepared according to the reaction as set forth for the purposes of the description in Scheme 1:

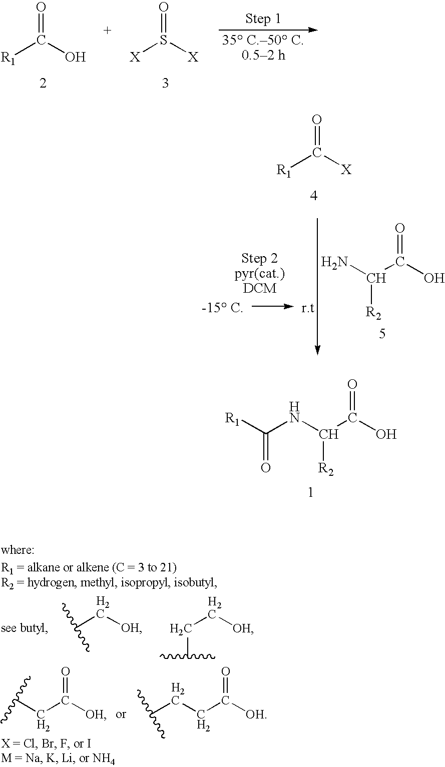

With reference to Scheme 1, in Step 1 an acyl halide (4) is produced via reaction of a fatty acid (2) with a thionyl halide (3).

In various embodiments of the present invention, the fatty acid of (2) is selected from the saturated fatty acid group comprising butyric or butanoic acid, caproic or hexanoic acid, caprylic or octanoic acid, capric or decanoic acid, lauric or dodecanoic acid, myristic or tetradecanoic acid, palmitic or hexadecanoic acid, stearic or octadecanoic acid, arachidic or eicosanoic acid, and behenic or docosanoic acid.

In additional or alternative embodiments of the present invention, the fatty acid of (2) is selected from the unsaturated fatty acid group comprising oleic acid, linoleic acid, linolenic acid, arachidonic acid, palmitoleic acid, eicosapentaenoic acid, docosahexaenoic acid, and erucic acid.

Furthermore the thionyl halide of (3) is selected from the group consisting of fluorine, chlorine, bromine, and iodine, the preferred method using chlorine or bromine.

The above reaction proceeds under conditions of heat ranging between from about 35° C. to about 50° C. and stirring over a period from about 0.5 hours to about 2 hours during which time the gases sulfur dioxide and acidic gas, wherein the acidic gas species is dependent on the species of thionyl halide employed, are evolved. Preferably, the reactions proceed at about 50° C. for about 1.25 hours.

Step 2 describes the addition of the prepared acyl halide (3) to a suspension of an amino acid (5) in dichloromethane (DCM), in the presence of catalytic pyridine (pyr), to form the desired amino acid-fatty acid amide (1). The addition of the acyl halide takes place at temperatures between about −15° C. and about 0° C. and with vigorous stirring. Following complete addition of the acyl halide the reaction continues to stir and is allowed to warm to room temperature before the target amide compound is isolated, the amide compound being a creatine fatty acid compound.

In various embodiments, according to aforementioned, using the saturated fatty acids, a number of compounds are produced; examples include, but are not limited to: 2-butyramido-3-hydroxybutanoic acid, 2-hexanamido-3-methylpentanoic acid, 2-octanamidopentanedioic acid, 2-decanamido-4-methylpentanoic acid, 2-dodecanamidosuccinic acid, 3-hydroxy-2-tetradecanamidopropanoic acid, 2-palmitamidosuccinic acid, 4-methyl-2-stearamidopentanoic acid, 2-icosanamido-3-methylbutanoic acid, and 2-docosanamidoacetic acid.

In additional embodiments, according to aforementioned, using the unsaturated fatty acids, a number of compounds are produced; examples include, but are not limited to: 3-hydroxy-2-oleamidopropanoic acid, 4-methyl-2-(9Z,12Z)-octadeca-9,12-dienamidopentanoic acid, 2-(9Z,12Z,15Z)-octadeca-9,12,15-trienamidopropanoic acid, 3-hydroxy-2-(5Z,8Z,11Z,14Z)-icosa-5,8,11,14-tetraenamidobutanoic acid, (Z)-2-hexadec-9-enamido-3-methylpentanoic acid, 2-(5Z,8Z,11Z,14Z,17Z)-icosa-5,8,11,14,17-pentaenamidopropanoic acid, 2-(4Z,7Z,10Z,13Z,16Z,19Z)-docosa-4,7,10,13,16,19-hexaenamidoacetic acid, and (Z)-3-methy-2-tricos-14-enamidobutanoic acid.

The following examples illustrate specific creatine-fatty acids and routes of synthesis thereof. One of skill in the art may envision various other combinations within the scope of the present invention, considering examples with reference to the specification herein provided.

### EXAMPLE 1

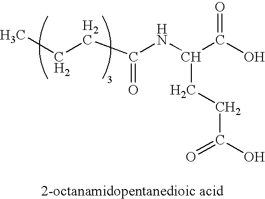

In a dry 2-necked, round bottomed flask, equipped with a magnetic stirrer and fixed with a separatory funnel, containing 10.07 ml (130 mmol) of thionyl bromide, and a water condenser, is placed 10.30 ml (65 mmol) of octanoic acid. Addition of the thionyl bromide is completed with heating to about 50° C. over the course of about 50 minutes. When addition of the thionyl bromide is complete the mixture is heated and stirred for an additional hour. The water condenser is then replaced with a distillation side arm condenser and the crude mixture is distilled. The crude distillate in the receiving flask is then fractionally distilled to obtain the acyl bromide, octanoyl bromide. This acyl bromide, 4.88 g (30 mmol), is put into a dry separatory funnel and combined with 50 ml of dry dichloromethane for use in the next step of the reaction.

In a dry 3-necked, round bottomed flask, equipped with a magnetic stirrer, a thermometer, a nitrogen inlet tube and the dropping funnel containing the octanoyl bromide solution, 7.94 g (54 mmol) of Glutamic acid is suspended, with stirring, in 150 ml of dry dichloromethane. To this suspension a catalytic amount (0.1 mmol) of pyridine is also added. The suspension is stirred in a dry ice and acetone bath to a temperature of between to about −10° C. and 0° C. When the target temperature is reached the drop wise addition of octanoyl bromide is commenced. Addition of octanoyl bromide continues, with cooling and stirring, until all of the octanoyl bromide is added, after which the reaction is allowed to warm to room temperature with constant stirring. The solution is then filtered to remove any remaining Glutamic acid and the volatile dichloromethane and pyridine are removed under reduced pressure yielding 2-octanamidopentanedioic acid.

### EXAMPLE 2

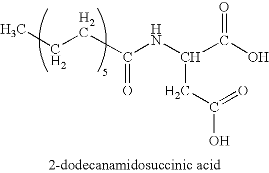

In a dry 2-necked, round bottomed flask, equipped with a magnetic stirrer and fixed with a separatory funnel, containing 13.13 ml (180 mmol) of thionyl chloride, and a water condenser, is placed 20.03 g (100 mmol) of dodecanoic acid. Addition of the thionyl chloride is completed with heating to about 45° C. over the course of about 30 minutes. When addition of the thionyl chloride is complete the mixture is heated and stirred for an additional 45 minutes. The water condenser is then replaced with a distillation side arm condenser and the crude mixture is distilled. The crude distillate in the receiving flask is then fractionally distilled to obtain the acyl chloride, dodecanoyl chloride. This acyl chloride, 7.66 g (35 mmol), is put into a dry separatory funnel and combined with 50 ml of dry dichloromethane for use in the next step of the reaction.

In a dry 3-necked, round bottomed flask, equipped with a magnetic stirrer, a thermometer, a nitrogen inlet tube and the dropping funnel containing the dodecanoyl chloride solution, 7.45 g (56 mmol) of Aspartic acid is suspended, with stirring, in 150 ml of dry dichloromethane. To this suspension a catalytic amount (0.1 mmol) of pyridine is also added. The suspension is stirred in a dry ice and acetone bath to a temperature of between about −15° C. and 0° C. When the target temperature is reached the drop wise addition of dodecanoyl chloride is commenced. Addition of dodecanoyl chloride continues, with cooling and stirring, until all of the dodecanoyl chloride is added, after which the reaction is allowed to warm to room temperature with constant stirring. The solution is then filtered to remove any remaining Aspartic acid, and the volatile dichloromethane and pyridine are removed under reduced pressure yielding 2-dodecanamidosuccinic acid.

### EXAMPLE 3

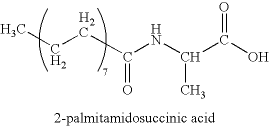

In a dry 2-necked, round bottomed flask, equipped with a magnetic stirrer and fixed with a separatory funnel, containing 7.75 ml (100 mmol) of thionyl bromide, and a water condenser, is placed 12.82 g (50 mmol) of palmitic acid. Addition of the thionyl bromide is completed with heating to about 50° C. over the course of about 50 minutes. When addition of the thionyl bromide is complete the mixture is heated and stirred for an additional hour. The water condenser is then replaced with a distillation side arm condenser and the crude mixture is distilled. The crude distillate in the receiving flask is then fractionally distilled to obtain the acyl bromide, palmitoyl bromide. This acyl bromide, 16.02 g (50 mmol), is put into a dry separatory funnel and combined with 75 ml of dry dichloromethane for use in the next step of the reaction.

In a dry 3-necked, round bottomed flask, equipped with a magnetic stirrer, a thermometer, a nitrogen inlet tube and the dropping funnel containing the palmitoyl bromide solution, 5.34 g (60 mmol) of Alanine is suspended, with stirring, in 150 ml of dry dichloromethane. To this suspension a catalytic amount (0.1 mmol) of pyridine is also added. The suspension is stirred in a dry ice and acetone bath to a temperature of between to about −10° C. and 0° C. When the target temperature is reached the drop wise addition of palmitoyl bromide is commenced. Addition of palmitoyl bromide continues, with cooling and stirring, until all of the palmitoyl bromide is added, after which the reaction is allowed to warm to room temperature with constant stirring. The solution is then filtered to remove any remaining Alanine and the volatile dichloromethane and pyridine are removed under reduced pressure yielding 2-palmitamidosuccinic acid.

### EXAMPLE 4

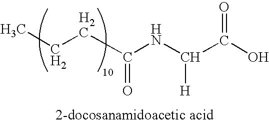

In a dry 2-necked, round bottomed flask, equipped with a magnetic stirrer and fixed with a separatory funnel, containing 7.88 ml (108 mmol) of thionyl chloride, and a water condenser, is placed 20.44 g (60 mmol) of docosanoic acid. Addition of the thionyl chloride is completed with heating to about 45° C. over the course of about 30 minutes. When addition of the thionyl chloride is complete the mixture is heated and stirred for an additional 70 minutes. The water condenser is then replaced with a distillation side arm condenser and the crude mixture is distilled. The crude distillate in the receiving flask is then fractionally distilled to obtain the acyl chloride, docosanoyl chloride. This acyl chloride, 21.60 g (60 mmol), is put into a dry separatory funnel and combined with 100 ml of dry dichloromethane for use in the next step of the reaction.

In a dry 3-necked, round bottomed flask, equipped with a magnetic stirrer, a thermometer, a nitrogen inlet tube and the dropping funnel containing the docosanoyl chloride solution, 7.20 g (96 mmol) of Glycine is suspended, with stirring, in 150 ml of dry dichloromethane. To this suspension a catalytic amount (0.1 mmol) of pyridine is also added. The suspension is stirred in a dry ice and acetone bath to a temperature of between about −15° C. and 0° C. When the target temperature is reached the drop wise addition of docosanoyl chloride is commenced. Addition of docosanoyl chloride continues, with cooling and stirring, until all of the docosanoyl chloride is added, after which the reaction is allowed to warm to room temperature with constant stirring. The solution is then filtered to remove any remaining Glycine, and the volatile dichloromethane and pyridine are removed under reduced pressure yielding 2-docosanamidoacetic acid.

### Example 5

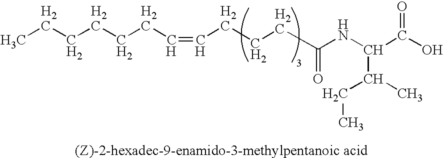

In a dry 2-necked, round bottomed flask, equipped with a magnetic stirrer and fixed with a separatory funnel, containing 13.13 ml (180 mmol) of thionyl chloride, and a water condenser, is placed 25.44 ml (100 mmol) of palmitoleic acid. Addition of the thionyl chloride is completed with heating to about 40° C. over the course of about 30 minutes. When addition of the thionyl chloride is complete the mixture is heated and stirred for an additional 55 minutes. The water condenser is then replaced with a distillation side arm condenser and the crude mixture is distilled. The crude distillate in the receiving flask is then fractionally distilled to obtain the acyl chloride, (Z)-hexadec-9-enoyl chloride. This acyl chloride, 11.55 g (40 mmol), is put into a dry separatory funnel and combined with 75 ml of dry dichloromethane for use in the next step of the reaction.

In a dry 3-necked, round bottomed flask, equipped with a magnetic stirrer, a thermometer, a nitrogen inlet tube and the dropping funnel containing the (Z)-hexadec-9-enoyl chloride solution, 8.39 g (64 mmol) of Isoleucine is suspended, with stirring, in 150 ml of dry dichloromethane. To this suspension a catalytic amount (0.1 mmol) of pyridine is also added. The suspension is stirred in a dry ice and acetone bath to a temperature of between about −15° C. and 0° C. When the target temperature is reached the drop wise addition of (Z)-hexadec-9-enoyl chloride is commenced. Addition of (Z)-hexadec-9-enoyl chloride continues, with cooling and stirring, until all of the (Z)-hexadec-9-enoyl chloride is added, after which the reaction is allowed to warm to room temperature with constant stirring. The solution is then filtered to remove any remaining Isoleucine, and the volatile dichloromethane and pyridine are removed under reduced pressure yielding (Z)-2-hexadec-9-enamido-3-methylpentanoic acid.

Thus while not wishing to be bound by theory, it is understood that reacting an amino acid or derivative thereof with a fatty acid or derivative thereof to form an amide can be used enhance the bioavailability of the amino acid or derivative thereof by improving stability of the amino acid and by increasing solubility and absorption. Furthermore, it is understood that, dependent upon the specific fatty acid, for example, saturated fatty acids form straight chains allowing mammals to store chemical energy densely, or derivative thereof employed in the foregoing synthesis, additional fatty acid-specific benefits, separate from the amino acid substituent, will be conferred.

### Extension and Alternatives

In the foregoing specification, the invention has been described with a specific embodiment thereof; however, it will be evident that various modifications and changes may be made thereto without departing from the broader spirit and scope of the invention.

What is claimed:

1. A compound having the general structure: 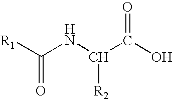
wherein R₁ is selected from the group consisting of alkanes and alkenes;
said alkanes and alkene having from 3 to 21 carbons; 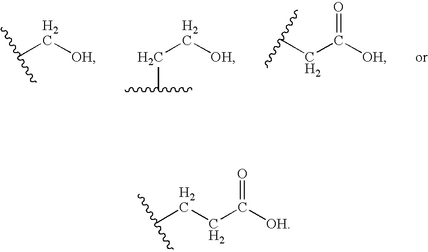
wherein R₂ is hydrogen, methyl, isopropyl, isobutyl, sec butyl,

2. The compound according to claim 1 wherein R₁ is an alkane having 3 to 5 carbons.

3. The compound according to claim 1 wherein R₁ is an alkane having 7 to 9 carbons.

4. The compound according to claim 1 wherein R₁ is an alkane having 11 to 13 carbons.

5. The compound according to claim 1 wherein R₁ is an alkane having 15 to 17 carbons.

6. The compound according to claim 1 wherein R₁ is an alkane having 19 to 21 carbons.

7. The compound according to claim 1 wherein R₁ is an alkene having at least one carbon-carbon double bond, comprising 3 to 5 carbons.

8. The compound according to claim 1 wherein R₁ is an alkene having at least one carbon-carbon double bond, comprising 7 to 9 carbons.

9. The compound according to claim 1 wherein R₁ is an alkene having at least one carbon-carbon double bond, comprising 11 to 13 carbons.

10. The compound according to claim 1 wherein R₁ is an alkene having at least one carbon-carbon double bond, comprising 15 to 17 carbons.

11. The compound according to claim 1 wherein R₁ is an alkene having at least one carbon-carbon double bond, comprising 17 to 21 carbons.
